(19) United States
(12) Patent Application Publication
(10) Pub. No.: US 2018/0078270 A1
(43) Pub. Date: Mar. 22, 2018

(54) SYSTEMS, METHODS AND DEVICES THAT FACILITATE MECHANICAL SINUS DILATION

(71) Applicant: Mark M. SCHEURER, Coronado, CA (US)

(72) Inventor: Mark M. SCHEURER, Coronado, CA (US)

(21) Appl. No.: 15/706,675

(22) Filed: Sep. 16, 2017

Related U.S. Application Data
(60) Provisional application No. 62/396,183, filed on Sep. 18, 2016.

(51) Int. Cl. A61B 17/24; A61M 29/00; A61M 25/00

(52) U.S. Cl. CPC A61B 17/24; A61M 2029/025; A61M 25/0041; A61M 29/00

(57) ABSTRACT

A system, method and apparatus are provided for facilitating mechanical sinus ostial dilation in a medical setting with a device that is formed of all sterilizable constituent components. A reusable mechanical sinus dilation device is provided that is autoclavable replacing a fluid-filled balloon or bladder, i.e. a balloon/hydraulic mechanism, with mechanical structures for dilation. The mechanical sinus dilation device incorporates a radially expanding mechanical (structural) dilation mechanism. The removal of all components formed of non-autoclavable materials, e.g., plastic pieces, permits embodiments to be autoclaved, or otherwise sterilized by a combination of heat and pressure, so as to render each individual mechanical sinus dilation device according to the disclosed embodiments reusable across some plurality of sinus dilation procedures. A dilating sleeve circumferentially surrounds the radially extending movable mechanical structures in a configuration may provide an opportunity to exchange dilating sleeves between sinus dilation procedures or operations.

This application claims the benefit of U.S. Provisional Patent Application No. 62/296,183, entitled “Mechanical Sinus Dilation Device,” by Mark M. Scheurer, filed Sep. 18, 2016, the disclosure of which is hereby incorporated by reference in its entirety.

### BACKGROUND

### 1. Field of the Disclosed Embodiments

This disclosure is directed to a uniquely reusable system and/or apparatus for facilitating mechanical sinus ostial dilation or eustachian tube dilation in a medical setting with a device that is formed of all sterilizable constituent components.

### 2. Related Art

The paranasal sinuses are spaces in the facial skeleton above, below, between, and behind the eyes, which are normally filled with air and are connected to the nasal cavity. These sinuses continually produce mucus, which drains into the nasal cavity through small passageways called ostia. These ostia also allow the free flow of air between the nose and sinuses. Both ventilation and unrestricted mucus drainage are critical for sinus health. Sinusitis refers generally to a class of medical conditions involving inflammation of the sinus cavities and sinus openings within the nose. Sinusitis is generally categorized according to the length of time the conditions are present and how often the conditions recur.

Acute sinusitis is characterized by a short duration of symptoms, and usually does not require medical intervention.

Chronic sinusitis describes sinus inflammation that persists for 12 weeks or longer. Chronic sinusitis, or repeated episodes of acute sinusitis, may require treatment with anti-inflammatory medications, antibiotics, or saline rinses. Recurrent or chronic sinusitis is often associated with narrowing of the passageways that drain and ventilate the sinuses. In cases that do not respond to appropriate medical therapy, surgical intervention to enlarge these passageways has been recommended for several decades and is generally very effective.

The central goal of surgical therapy is the improvement of sinus drainage and ventilation. Enlargement of the ostia or passageways between the affected sinuses and the nasal cavity facilitates this goal. Sinus procedures have evolved from external approaches involving incisions through the facial skin and oral cavity to those employing nasal endoscopes, which allow sinus ostial enlargement from entirely within the nasal cavity.

Endoscopic procedures have traditionally involved locating the narrowed sinus ostia within the nasal cavity and then using cutting instruments to enlarge the openings as much as possible, beyond what is ever seen in normal anatomy. More recently, evidence has shown that the use of balloons to dilate sinus ostia to the smaller dimensions seen in normal anatomy is less invasive and traumatic, involves a much shorter recovery time, and is equally as effective as traditional surgery. Additionally, these procedures can be performed in an office under local anesthesia, substantially reducing costs compared to the use of an operating room. Sinus dilation devices are generally configured with small balloons or bladders to be filled with water, saline or other biologically-neutral fluid and are made of disposable, non-sterilizable plastic.

Even newer procedures involve the use of sinus-type balloons to dilate the eustachian tubes. These structures are passageways similar to the ostia of sinuses in that they connect the middle ear spaces to the nasal cavity, thereby allowing air exchange and ventilation of those spaces. A middle ear space can be considered a type of paranasal sinus. It is normally filled with air, and air exchange normally occurs between that space and the nasal cavity. Normal eustachian tube function is critical for this air exchange; the root cause of most chronic ear infections is eustachian tube dysfunction with consequent poor middle ear space ventilation. Balloon dilation of the eustachian tubes has been found to be very effective in improving ventilation of the middle ear spaces when eustachian tube function is suboptimal. Descriptions of the device disclosed mainly discuss its use for the paranasal sinuses. Embodiments of the device may have other surgical applications, however, such as dilation of the eustachian tubes discussed above. Accordingly, examples of use provided in this disclosure should not be considered limiting in any way with regard to any particular surgical application.

FIGS. 1 and 2 illustrate schematic diagrams of an exemplary embodiment of a simple conventional balloon sinus dilation device 100,200 in a simplest form for ease of understanding. In FIGS. 1 and 2, like numbers refer to like components of the illustrated exemplary embodiments. As shown in FIGS. 1 and 2, the conventional sinus dilation device 100,200 generally includes a main body component 150,250, which may be in a form of a handle (simply or ergonomically designed) to facilitate manipulation of the exemplary conventional balloon sinus dilation device 100,200 in operation.

A conventional balloon sinus dilation device 100,200 includes a central rod-like or wire structure 110 (a like element to element 110 in FIG. 1 is obscured in FIG. 2, as it is in operation, as being generally within the other structural components described below) that the surgeon may bend in almost any manner desired in order to facilitate penetration of the blocked, or otherwise occluded, sinus passage, cavity or opening, in use, and over which the balloon or bladder structure may be advanced.

A balloon, bladder or balloon-like structure 120,220 is then provided encircling the central rod-like or wire structure 110. An intervening structure, which may be in the form of a hub 130,230, is generally provided as an interface between the balloon, bladder or balloon-like structure 120,220, and a cannula 140,240 through which fluid will be passed to “inflate” the balloon, bladder or balloon-like structure 120,220 in a radially outward direction in use.

A sliding component 160,260 is provided in a cooperating groove 170,270 in the main body component 150,250, and structurally attached to the cannula 140,240. The sliding component 160,260 is moved along the groove 170,270 in the main body component 150,250 in direction “A” in order to advance a combination of (1) the cannula 140,240, (2) the hub 130,230, and (3) ultimately the balloon, bladder or balloon-like structure 120,220, along the central rod-like or wire structure 110 from a first (or rest or non-engaged) position as shown in FIG. 1 to a second (or operating or engaging) position as shown in FIG. 2.

Once advanced to the second/operating/engaging position, as shown in FIG. 2, such that the initially deflated balloon, bladder or balloon-like structure 120,220 is advanced along the central rod-like or wire structure 110 into the sinus passage, cavity or opening to be dilated, the balloon, bladder or balloon-like structure 120,220 is positioned in the “blocked” sinus passage, cavity and/or opening in a manner similar to a procedure, for example, for insertion of a balloon into an occluded artery in the commonly-known balloon angioplasty medical procedure.

Once the balloon, bladder or balloon-like structure 120,220 is positioned in the blocked sinus passage, cavity or opening, the balloon, bladder or balloon-like structure 120,220 is then “inflated” by displacing/advancing fluid from a fluid reservoir 180,280 through the cannula 140,240 past the hub 130,230 and into the balloon, bladder or balloon-like structure 120,220. The translation of the fluid along the described flow path thereby causes the balloon, bladder or balloon-like structure 120,220 to expand in a radially outward direction with respect to the central rod-like or wire structure (see element 110 in FIG. 1). As shown in FIG. 2, the translation of the fluid from the fluid reservoir 280 may be facilitated by exerting pressure, for example, against an outer surface of a flexible or malleable fluid reservoir 280 in direction B. In this manner, the fluid in the fluid reservoir 280 is made to flow along the flow path described above to expand the balloon, bladder or balloon-like structure 120,220 to its radially outward extent in the manner generally depicted in FIG. 2. In embodiments, the fluid reservoir 180,280 may be alternatively in a form of a syringe or other commonly-known structure by which fluid, including water, saline or other like fluid, may be held at rest in the fluid reservoir, and ejected from the fluid reservoir directly, or through intervening structures, along the flow path described above.

The expanding balloon, bladder or balloon-like structure 120,220 exerts outward radial pressure against internal walls of the sinus passages, sinus cavities and/or sinus ostial openings in a manner that gently, yet generally irreversibly, expands or dilates those passages, cavities or openings in a radially outward direction. Once the expansion procedure is complete, the steps of the process may be reversed in order to evacuate the fluid from the balloon, bladder or balloon-like structure 120,220. In this manner, an outer surface of the balloon, bladder or balloon-like structure 120,220 may then be disengaged from the newly-expanded (dilated) inner walls of the sinus passages, sinus cavities and/or sinus ostial openings, and the conventional balloon sinus dilation device 100,200 extracted from the passages, cavities or openings to complete the procedure.

Sinus dilation devices such as those described generally above are available in a number of configurations from a variety of medical device manufacturers. In general, these devices are single-use components. The inclusion of fluid reservoirs, and a construction in which at least parts of these devices are formed of materials that generally cannot withstand high temperatures, render these devices non-autoclavable, and therefore not medically reusable. The inability to sterilize and reuse conventional sinus dilation devices such as those described generally above increases the per-procedure price to individual patients, and often requires additional personnel and set up to accomplish the sinus dilation procedure.

These drawbacks are generally accepted as “costs” of doing business with regard to sinus dilation procedures. With the costs of all medical procedures having come under increasing scrutiny in the last several years, some manner by which to decrease the per-procedure costs with respect to sinus dilation procedures may prove beneficial.

### SUMMARY

Based on the foregoing, it would be advantageous to provide a sinus dilation device that may be specifically configured to provide substantially the same procedural benefits of the conventional balloon sinus dilation device in a component system that would be autoclavable, and therefore reusable across a number of sinus dilation procedures.

Exemplary embodiments of the systems, methods and devices according to this disclosure may provide a reusable mechanical sinus dilation device that is autoclavable and does not suffer from the shortfalls described generally above.

Exemplary embodiments may provide a sinus dilation device that does not use a fluid-filled balloon or bladder, i.e. a balloon/hydraulic mechanism, for dilation.

In embodiments, the disclosed mechanical sinus dilation device may incorporate a radially expanding mechanical (structural) dilation mechanism. The removal of all components formed of non-autoclavable materials, e.g., plastic pieces, may permit embodiments according to the disclosed mechanical sinus dilation device to be autoclaved, or otherwise sterilized by a combination of heat and pressure, so as to render each individual mechanical sinus dilation device according to the disclosed embodiments reusable across some plurality of sinus dilation procedures.

In embodiments, the disclosed mechanical dilation mechanism, replacing the fluid-filled balloon or bladder at a distal end of the exemplary mechanical sinus dilation device, may simplify the procedure for sinus dilation to essentially a single “trigger-like” or “scissor-like” operation. In embodiments, procedures undertaken employing the disclosed mechanical dilation mechanism may be of further advantage in that disclosed mechanical sinus dilation devices may be faster and easier to use during surgery than a conventional balloon-type sinus dilation device typically relying on inflation with water or other fluid. Embodiments of the disclosed mechanical sinus dilation device may simplify the sinus dilation procedure in a manner that would not require an additional person to accomplish, or assist in, the typical outpatient sinus dilation procedure.

In embodiments, a distal end of an exemplary mechanical sinus dilation device may be partially, discretely, or continuously comprised, over a particular axial length, of an external sleeve. The external sleeve may be formed of an autoclavable material including, but not limited to, a braided metal sleeve, a Kevlar® sleeve or a sleeve of a similar highly-heat resistant, yet expandable material.

In embodiments, an objective of a selection of a material for an external radially expanding sleeve along at least a portion of an axial length of a distal end of the disclosed exemplary mechanical sinus dilation devices would be to provide such material with inherent strength and pliability, the expandable sleeve being formed in a configuration so as to facilitate expansion and retraction of the expandable sleeve in the radial direction across a number of procedures thereby rendering the device reusable once sterilized between procedures.

In embodiments, inclusion of an external radially expanding sleeve along at least a portion of an axial length of the distal end of the disclosed exemplary mechanical sinus dilation device may ensure that outward radial pressure exerted by the device along an inner wall of the sinus passage, sinus cavity, and/or sinus ostial opening may be axially disposed in a manner similar to the conventional balloon-type expansion component in order that an adequate linear portion of the inner wall of the passage, cavity or opening is outwardly repositioned in the dilation procedure.

Exemplary embodiments may include a number of movable mechanical structures radially extending from a distal end of the exemplary mechanical sinus dilation device at least across a certain axial length of the distal end of the device covered by an expandable sleeve. Radially extending movable mechanical structures may extend from an axis of the device in one or more of a linear (along the axis) expansion and a helical (rotated about the axis) expansion, or in any other like manner that may provide mechanical radially directed force against an inner surface of an expandable sleeve.

In embodiments, movable mechanical structures extending radially outward from a distal end of the exemplary mechanical sinus dilation device may be usable to radially outwardly expand an expandable sleeve around the distal end of the exemplary sinus dilation device and covering the movable mechanical structures as deployable rigid extendable supports underneath the sleeve.

In embodiments, specific extension mechanisms for the radially outwardly movable mechanical structures may be similar to those commonly seen with other medical instruments such as the sinus backbiter in which the deployed component is raised from a flush surface by movement of the surgeon's hand on the handle of the instrument.

In embodiments, the radially extending movable mechanical structures may be moved or extended in the radially outward direction through activation of intermediary actuating structures to extend the radially extending movable mechanical structures to a deployed position. In embodiments, this actuation may occur through action of a surgeon's hand holding an exemplary embodiment of the disclosed mechanical sinus dilation device in a manner similar to a sinus backbiter.

In embodiments, activation of the exemplary radially extending movable mechanical structures in a dilation mechanism may be accomplished through a standard two-ringed scissor-type handle arrangement.

In embodiments, the disclosed sinus dilation device may employ an internal (or axially central) rod or wire within a sheath, the sheath generally containing the radially extending movable mechanical structures.

In embodiments, such an internal (or axially central) rod or wire may be mechanically connected to actuators such that movement of the rod or wire would deploy the actuators or movable mechanical structures associated with the actuators from a position substantially flush with an external surface of the sheath.

In embodiments, movement of an internal (or axially central) rod or wire may be reversible in order that radially extending movable mechanical structures and/or actuators may be positively mechanically retracted.

In embodiments, a dilating sleeve may circumferentially surround the sheath or other inner structure containing the radially extending movable mechanical structures or actuators in a manner that, at rest, the radially extending movable mechanical structures or actuators are retracted so as to present a substantially flush outer surface with respect to the sheath. Such a configuration may provide an opportunity to exchange dilating sleeves between sinus dilation procedures or operations.

In embodiments, radial extension of the movable mechanical structures, and/or radial deployment of actuators, is envisioned as occurring in a manner whereby an expandable and circumferential sleeve segment may be rigidly tented open by the radially extended mechanical movable structures or actuators when exerting radially outward force against an inner wall of a sinus passage, sinus cavity and/or sinus ostial opening.

In embodiments, an axial length of a dilating sleeve along a length of the distal end of the disclosed sinus dilating device may achieve rigid deployment by being supported by a plurality of radially extending movable mechanical components. An axial length of the dilating sleeve may be modified, i.e., shortened, during the dilation phase. As such, adequate slack may be provided in the dilating sleeve when in an at rest position in order that the dilating sleeve may reach full expansion under tension exerted by the radially extending movable mechanical components and/or deployed actuators. In embodiments, axial shortening of a dilating sleeve may be achieved passively by deployment of the radially extending movable mechanical structures and/or actuators.

In other embodiments, a dilating sleeve may be actively shortened by an additional structure that may be in a form of an outside sliding of fixed sleeve mechanism connected to the dilating sleeve, which may extend to a handle of an exemplary embodiment of the disclosed mechanical sinus dilation device.

In embodiments, activation of a handle of the disclosed mechanical sinus dilation device according to any known configuration may simultaneously deploy both radially extending movable mechanical structures and/or actuators, and provide for mechanical shortening of the expandable sleeve such that the expandable or dilating sleeve would be fully expanded under tension from the radially extending movable mechanical structures and/or actuators. In embodiments, an opposite or counter movement of the handle of the disclosed mechanical sinus dilation device may positively mechanically retract the radially extending movable mechanical structures and/or actuators, and pull back on the expandable or dilating sleeve to restore the expandable or dilating sleeve of the disclosed mechanical sinus dilation device to its non-deployed (or unexpanded), thinner configuration.

In embodiments, the dilating sleeve may be held in a non-deployed, non-dilated or non-radially-extended position by a spring anchored more proximally (toward the handle) on an outer part of the disclosed mechanical sinus dilation device. In this manner, the dilating segment or sleeve may be positively mechanically and/or automatically retracted against an outer surface of the distal end of the disclosed mechanical sinus dilation device unless the dilating sleeve is expressly being forced open by the movement of the radially extending movable mechanical structures/members and/or actuators.

In embodiments, a distal portion of the disclosed mechanical sinus dilation device may be linear, i.e., a straight terminal portion, or otherwise may be pre-curved or malleable enough by a surgeon to be positioned at substantially any acute angle with respect to an axis of the device in general or otherwise according to any curvature chosen by the surgeon to facilitate the insertion of the distal portion of the disclosed mechanical sinus dilation device into a sinus passage, sinus cavity and/or sinus ostial opening to be dilated during a sinus dilation procedure employing the disclosed device.

These and other features, and advantages, of the disclosed systems, methods and devices are described in, or apparent from, the following detailed description of various exemplary embodiments.

### BRIEF DESCRIPTION OF THE DRAWINGS

Various exemplary embodiments of the disclosed unique reusable system and/or apparatus for facilitating mechanical sinus dilation in a medical setting will be described, in detail, with reference to the following drawings, in which:

FIGS. 1 and 2 illustrate schematic diagrams of an exemplary embodiment of a simple conventional balloon sinus dilation device in a simplest form for ease of understanding of the subject matter of this disclosure;

FIGS. 3A and 3B illustrate an exemplary embodiment of an overall view of a mechanical sinus dilation device according to this disclosure, respectively in a first (non-dilated) condition (FIG. 3A) and in a second (dilated) condition (FIG. 3B), and FIG. 3C illustrates an exemplary variation in an operating mechanism for the overall view of a mechanical sinus dilation device shown in FIG. 3A and FIG. 3B;

FIG. 4 illustrates a side view of detail of a first exemplary distal portion of a mechanical sinus dilation device according to this disclosure, with any dilating sleeve removed for clarity;

FIG. 5 illustrates a side view of detail of the first exemplary distal portion of the mechanical sinus dilation device shown in FIG. 4 according to this disclosure with radially extending movable mechanical structures or actuators raised out of a first (non-dilated) position to a second (dilated) position in which the radially extending movable mechanical structures or actuators tent open an expandable sleeve according to this disclosure;

FIG. 6 illustrates an end view of detail of the first exemplary distal portion of the mechanical sinus dilation device shown in FIGS. 4 and 5 according to this disclosure with the radially extending mechanical structures or actuators raised to the second position;

FIG. 7 illustrates a side view of detail of a second exemplary embodiment of a mechanical sinus dilation device according to this disclosure with an expandable sleeve overlying the radially extending movable mechanical structures or actuators in a first (non-dilated) position;

FIG. 8 illustrates a side view of detail of the second exemplary embodiment of the mechanical sinus dilation device shown in FIG. 7 according to this disclosure with the expandable sleeve overlying the radially extending movable mechanical structures or actuators in a second (dilated) position; and

FIG. 9 illustrates a flowchart of an exemplary method for employing a mechanical sinus dilation device according to this disclosure in a sinus dilation procedure.

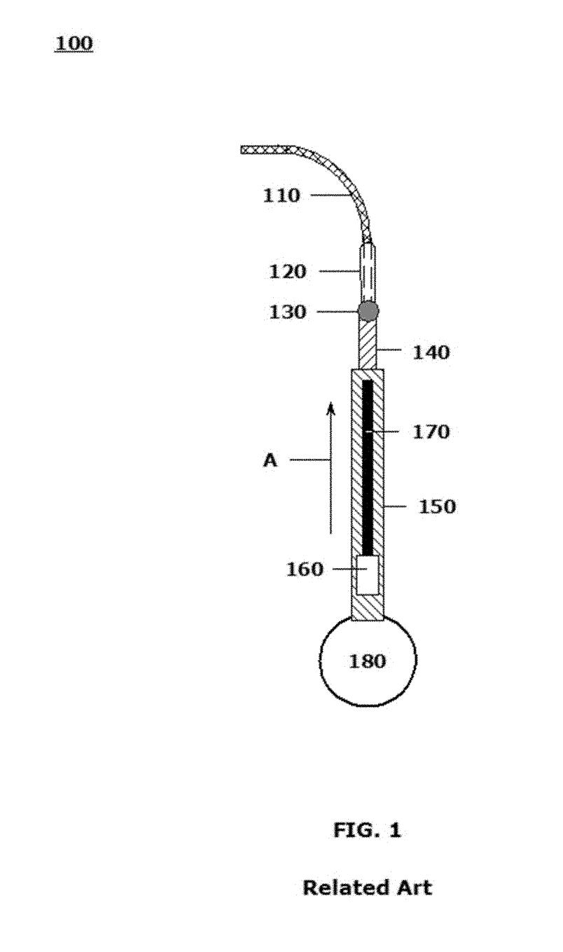

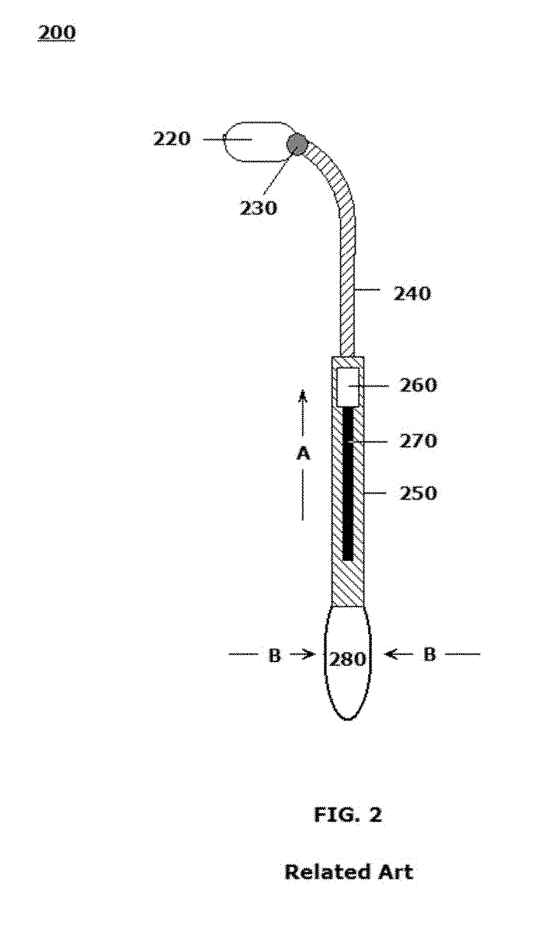

### DETAILED DESCRIPTION OF EMBODIMENTS

The disclosed unique reusable system and/or apparatus for facilitating mechanical sinus dilation in a medical setting will be described as being particularly usable for that purpose. This single real-world application for the system, method and apparatus according to this disclosure should, however, not be considered as limiting the disclosed embodiments to only that purpose. Rather, the disclosed embodiments are intended to provide an overview of a particular real-world scenario for employment of such a system, method and/or apparatus. Any advantageous employment of a similarly-configured device that may find utility in other medical procedures that may benefit from the teachings of the disclosed embodiments is intended to be encompassed by the detailed description that follows.

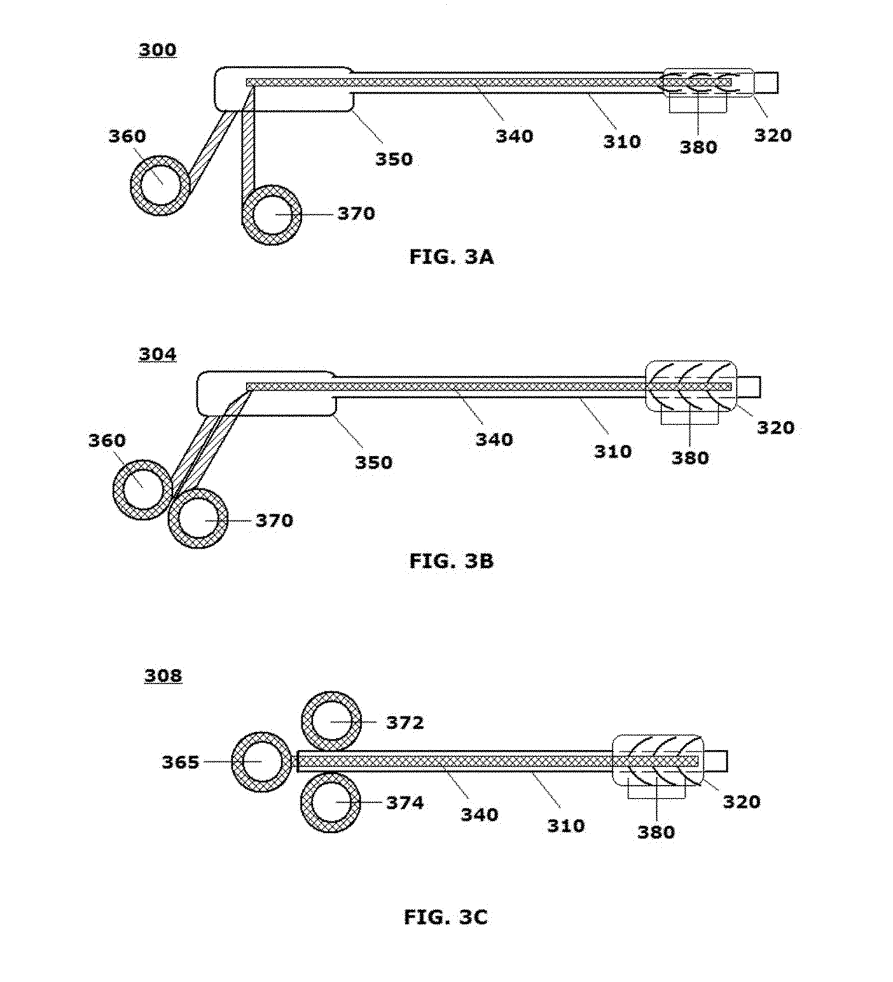

Reference will be made to a substantially integral device that may be of a configuration such as that shown in FIGS. 3A-3C, formed of, and/or incorporating components formed of, materials that are all heat resistant in order that the overall device may be sterilized, including via an autoclave, between multiple uses according to the described schemes, with temperatures exceeding 220° F. and pressures typically in a range of greater than 12 psi. The disclosed schemes may include techniques for configuring and employing such a device multiple times with a particular sterilization process in between each use to meet applicable medical standards for ensuring sterility, and to reduce the potential for contaminant transmission in medical employment scenarios.

Exemplary embodiments described and depicted in this disclosure should not be interpreted as being specifically limited to any particularly limiting material composition for the component structures of the disclosed device, including, but not limited to, the expandable dilation sleeve component, as will be described in greater detail below. Nor should the disclosed devices be considered to be limited to any particular method for forming the device as integral structures, or as structures of multiple connected components. Further, the exemplary embodiments described and depicted in this disclosure should not be interpreted as specifically limiting the configuration of any of the component parts, or to any limiting dimensions as may be implied by the detailed description that follows. All references to particular configurations and/or employment scenarios are intended to be illustrative only and are not intended to limit the disclosed concepts, compositions, processes, techniques, methods, uses, systems and devices in any manner. It should be recognized that any advantageous use of the disclosed structures and schemes for providing an advanced capability with respect to simplifying sinus dilation procedures, and reusing constituent components based on their ability to be autoclaved or sterilized between uses, that may advantageously employ systems, methods, techniques, and processes such as those discussed in detail in this disclosure is contemplated as being included within the scope of the disclosed exemplary embodiments.

In this regard, the disclosed systems, methods, apparatus and/or devices will be described as being particularly adaptable to the specific employment scenario of replacing a balloon-type sinus dilator for surgical intervention in a patient experiencing chronic sinusitis or other like condition, as described in the Background section of this disclosure. Again here, it must be noted that this description should not be considered limiting as to any intended use of a physical structure similar to that disclosed herein.

FIGS. 3A and 3B illustrate an exemplary embodiment of an overall view of a mechanical sinus dilation device 300,304, respectively in a first (non-dilated) condition 300 (FIG. 3A) and in a second (dilated) condition 304 (FIG. 3B). FIG. 3C illustrates an exemplary variation 308 in an operating mechanism for the overall view of a mechanical sinus dilation device 300,304 shown in FIG. 3A and FIG. 3B. As shown in FIGS. 3A, and 3B, the exemplary mechanical sinus dilation device 300,304 may include a thin body structure 310 extending substantially axially from a main body structure 350. The combination of the main body structure 350 and the thin body structure 310 may enclose, or otherwise encapsulate, an axial actuating structure 340, which may be in a form of a wire or rod-like structure, that is configured to be axially movable relative to the combination of the main body structure 350 and the thin body structure 310.

The actuating structure 340 may be usable to substantially connect at least a portion of a scissor-like set of components 360,370 (as an example of “actuating extensions”) with a plurality of radially extending movable mechanical dilating structures 380. The plurality of radially extending movable mechanical dilating structures 380 may be movable between a first (non-dilated) position (see FIG. 3A) in which a dilation sleeve 320 is substantially collapsed around the distal end of the thin body structure 310 and a second (dilated) position (see FIG. 3B) in which the dilation sleeve 320 is expanded by the plurality of radially extending movable mechanical dilating structures 380.

Movement of the combination of actuating structure 340 and the plurality of radially extending movable mechanical dilating structures 380 between the positions shown in FIGS. 3A and 3B, respectively, may be according to spreading apart and/or pinching together the scissor-like set of components 360,370. It should be noted that while depicted in FIGS. 3A and 3B in a particular matter that suggests that a first of the scissor-like components 360 may be fixedly attached to the main body structure 350, and the second of the scissor-like components 370 may be substantially movable relative to the main body structure 350 and the first of the scissor-like components 360, this is done for ease of understanding of a single non-limiting exemplary embodiment among myriad potential embodiments that may be contemplated. In other words, no particularly limiting configuration of a mechanism for controlling the axial movement of the actuating structure 340, and in turn the plurality of radially extending movable mechanical dilating structures 380, is intended to be implied by the depictions in FIGS. 3A and 3B. For example, the actuating structure 340 may be configured with a thumb loop 365 at a proximal end extending from a proximal end of the thin body structure 310. The thin body structure 310 may be configured with one or more finger loops or protrusions 372,374 extending substantially radially from a vicinity of the proximal end of the thin body structure 310. The thumb loop 365 and the one or more finger loops or protrusions 372,374 may constitute alternative examples of actuating extensions. In such a configuration, the movement of the combination of actuating structure 340 and the plurality of radially extending movable mechanical dilating structures 380 between the positions shown in FIGS. 3A and 3B, respectively, may be according to a “syringe-like” operation of the actuating structure 340 with respect to the thin body structure 310.

At least a distal portion of thin body structure 310 and the movable actuating structure 340, while shown as extending in a straight line substantially axially from the main body structure 350, may be bent at any acute angle, or otherwise according to any radius that will assist in an ability of an operator to advance the thin body structure 310 into an inflamed, blocked and/or occluded sinus passage with all of the distal end components of the device in the first (non-dilated) position shown in FIG. 3A.

Once a distal portion of the thin body structure 310 of the exemplary mechanical sinus dilation device 300,304,308 in use, is advanced into a patient's nasal cavity to an extent that at least the distal portion of the thin body structure 310, and specifically the dilation sleeve 320 is positioned in the inflamed, blocked and/or occluded sinus passage of the patient, actuation of the scissor-like set of components 360,370 (or the syringe-like actuation of the thumb loop 365 from an extended portion toward the finger loops, 372,374) between a first position and a second position may drive the actuating structure 340, and in turn the plurality of radially extending movable mechanical dilating structures 380. The induced movement in the plurality of radially extending movable mechanical dilating structures 380 may cause the dilation sleeve 320 to radially expand against an inner wall of the inflamed, blocked and/or occluded sinus passage in a manner that causes the passage to itself be dilated.

Once the dilation of the patient's sinus passage is acceptably completed, actuation of the scissor-like set of component 360,370 from the second position back to the first position may retract the actuating structure 340, and in turn the plurality of radially extending movable mechanical dilating structures 380. This retraction, particularly of the plurality of radially extending movable mechanical dilating structures 380 may cause the dilation sleeve 320 to collapse away from the inner wall of the now-dilated sinus passage in a manner that facilitates extraction of the exemplary mechanical sinus dilation device 300,304,308 from the sinus passage and patient's nasal cavity.

Each of the component body structures of the exemplary mechanical sinus dilation device 300,304,308, and the overall structure of the device itself, may be formed of one or more highly heat-resistant materials in order that the overall mechanical sinus dilation device 300,304,308 may be autoclavable, or otherwise sterilizable, between uses in order to facilitate multiple reuses of the device in a medical setting. For example, the dilation sleeve 320 may be of an expandable metal mesh, an expandable Kevlar®, or other like pliable but robust materials that may provide a capacity by which the dilation sleeve 320 may be routinely dilated and compressed in a repeated manner. In embodiments, the dilation sleeve 320 may be a single component of the overall exemplary mechanical sinus dilation device 300,304,308 that may be replaceable between uses.

FIG. 4 illustrates a first side view 400 of detail of a first exemplary distal portion of a mechanical sinus dilation device, with any dilation sleeve removed for clarity (see the dashed lined box). As shown in FIG. 4, the first exemplary distal portion may include a tip 410 for guiding the distal portion of the mechanical sinus dilation into a patient's inflamed, blocked and/or occluded sinus passage in the manner described above. As noted previously, while shown as a substantially linear structure, the first exemplary distal portion of the mechanical sinus dilation device may be bent in a manner that facilitates proper placement of a dilation sleeve in the patient's sinus passage. A plurality of radially extending (or extendable) movable mechanical dilating structures 480 may be provided in a substantially uniform manner encircling an entire circumference of the distal portion. In a first (non-dilated) position as shown in FIG. 4, the plurality of radially extending movable mechanical dilating structures 480 may be substantially flush with an outer body profile of the distal portion.

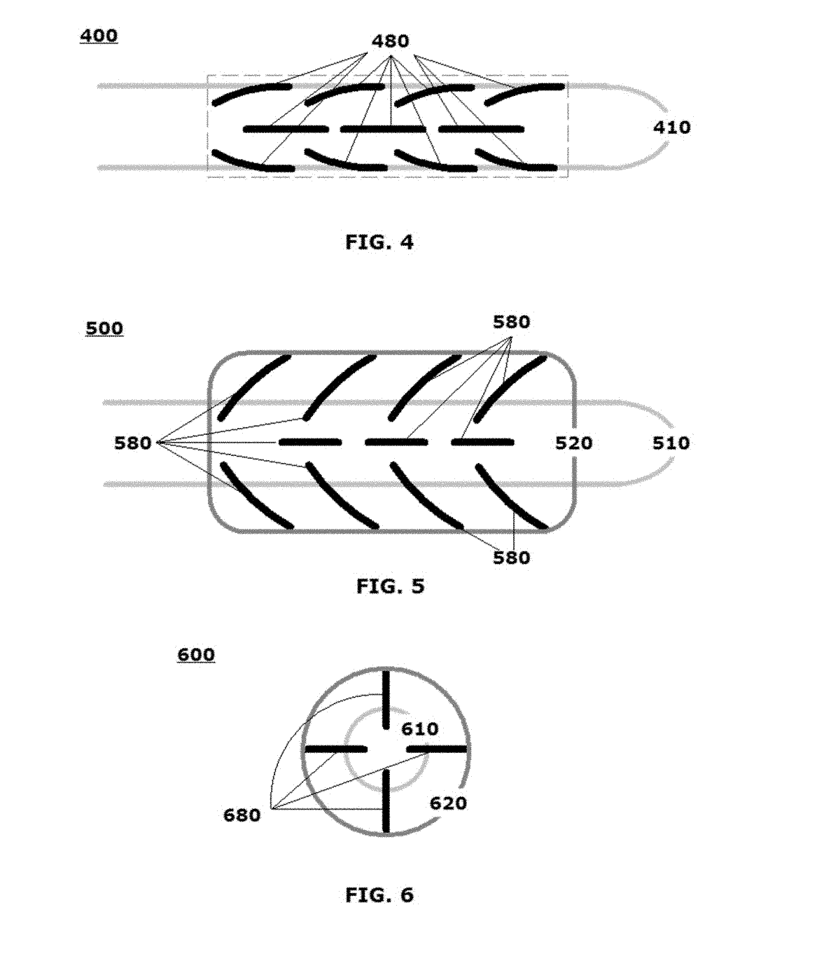

FIG. 5 illustrates a side view of detail of the first exemplary distal portion 500 of the mechanical sinus dilation device shown in FIG. 4 with the plurality of radially extending movable mechanical dilating structures 580 raised out of the first (non-dilated or flush) position shown in FIG. 4 to a second (dilated or extended) position once a distal tip 510 of the mechanical sinus dilation device is extended into a patient's inflamed, blocked and/or occluded sinus passage. In this second position, the plurality of radially extending movable mechanical dilating structures 580 may be substantially uniformly extended from a circumference of the distal portion in a manner that causes the dilating structures 580 to tent open an expandable dilation sleeve 520 substantially in the manner described above.

FIG. 6 illustrates an end view 600 of detail of the first exemplary distal portion of the mechanical sinus dilation device shown in FIGS. 4 and 5 with the plurality of radially extending movable mechanical dilating structures 680 raised to the second (dilated or extended) position. The detail shown in FIG. 6 reinforces that the plurality of radially extending movable mechanical dilating structures 680 extend substantially uniformly from a circumference of the tip of the distal portion 610 in the manner that causes the dilating structures 680 to tent open the expandable dilation sleeve 620.

As described above, the proposed mechanism to achieve rigid deployment/enlargement of the expandable dilation sleeve 620 in the radial direction would also potentially involve shortening the axial length of the dilation sleeve 620 during the dilation procedure. In this regard, adequate slack in the expandable dilation sleeve 620 may be available when the expandable dilation sleeve 620 is in its collapsed condition. The slack would be provided to substantially ensure that the expandable dilation sleeve 620 is unrestricted from reaching its full expansion of under tension when raised to the second (dilated) position through extension of the plurality of radially extending movable mechanical dilating structures 680. The resultant axial shortening of the dilation sleeve 620, when dilated, may be achieved passively simply as a result of extension of the plurality of radially extending movable mechanical dilating structures 680.

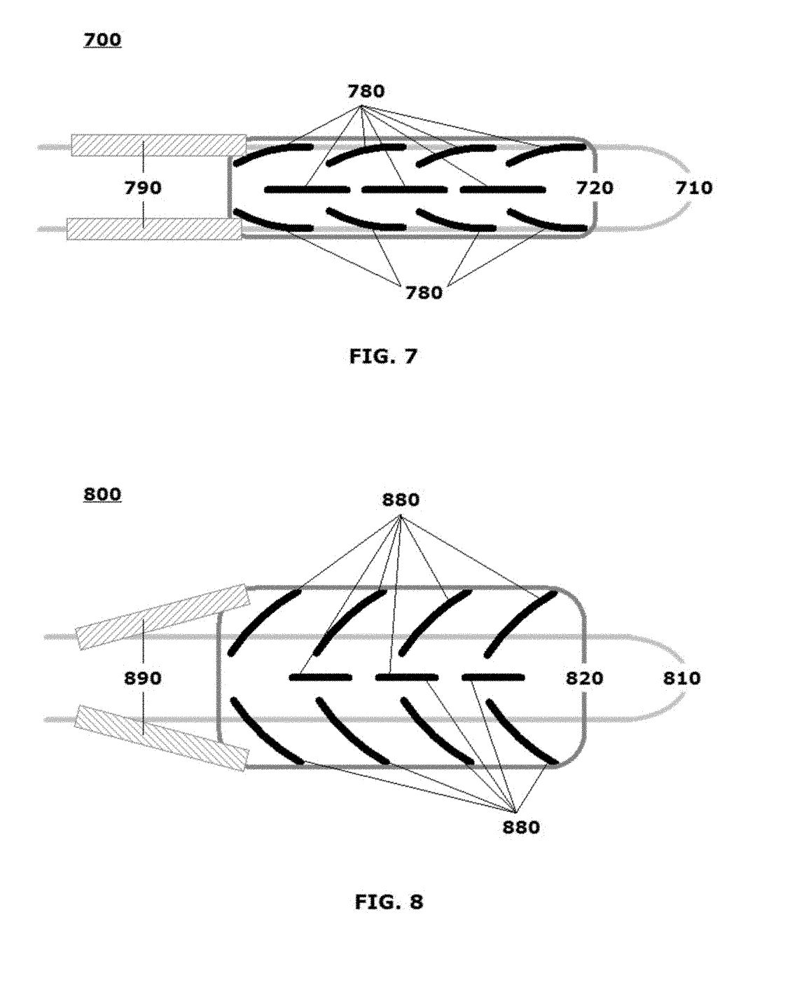

FIG. 7 illustrates a side view 700 of detail of a second exemplary embodiment of a mechanical sinus dilation device according to this disclosure with an expandable dilation sleeve 720 overlying a plurality of radially extending movable mechanical dilating structures 780 disposed just proximally to a tip 710 of the distal portion. As shown in FIG. 7, the expandable dilation sleeve 720 is collapsed around the distal portion and the plurality of radially extending movable mechanical dilating structures 780 in a first (non-dilated) position, substantially flush with an outer body structure of the distal portion, to facilitate advancing of the distal portion, guided by the distal tip 710 into the patient's sinus cavity.

As it is understood that, in operation, as the expandable dilation sleeve 720 is expanded radially, the expandable dilation sleeve 720 will tend to shorten in an axial direction, an extent of axial movement and/or shortening of the expandable dilation sleeve 720 in the axial direction may be actively controlled. In the embodiment shown in FIG. 7, one or more additional control structures 790 may be provided in a form of a plurality of discrete structures circumferentially placed around the distal portion of the mechanical sinus dilation device proximal to the expandable dilation sleeve 720, or alternatively in a form of a fixed or sliding sleeve mechanism encircling the distal portion of the mechanical sinus dilation device proximal to the expandable dilation sleeve 720, or in any other like configuration to carry into effect the below-described function of the one or more additional control structures 790.

Regardless of the particular structural composition of the one or more additional control structures 790, a distal end may be connected, adhered, or otherwise affixed to the expandable dilation sleeve 720. In embodiments, a proximal end of the one or more additional control structures 790 may extend to the handle of the mechanical sinus dilation device. In this manner, in operation, activation of the handle of the mechanical sinus dilation device may simultaneously control radial extension of the movable mechanical dilating structures 780 and control shortening of the expandable dilation sleeve 720 connected to the one or more additional control structures 790.

In the embodiment shown in FIG. 7, the one or more additional control structures 790 may be added discretely or continuously around a circumference of the distal portion of the mechanical sinus dilation device. The one or more additional control structures 790 may be fixedly or movably attached to the distal portion at a position proximal to the expandable dilation sleeve 720. The one or more additional control structures 790 may be usable to restrict and/or control an extent of the axial movement or shrinkage/elongation of the expandable dilation sleeve 720 in operation.

FIG. 8 illustrates a side view 800 of detail of the second exemplary embodiment of the mechanical sinus dilation device shown in FIG. 7 with the expandable sleeve overlying the radially extending mechanical dilating structures 880 in a second (dilated) position. Again here, one or more additional control structures 890, distal ends of which are connected to the expandable dilation sleeve 820, are provided to limit or otherwise control a shortening and/or elongation of the expandable dilation sleeve 820 in an axial direction in the course of dilation or compression of the expandable dilation sleeve 820. In embodiments in which a proximal end of the one or more additional control structures 890 may extend to the handle of the mechanical sinus dilation device, activation of the handle of the device may simultaneously control radial extension of the movable mechanical dilating structures 880 and control shortening of the expandable dilation sleeve 820 connected to the one or more additional control structures 890 as the expandable dilation sleeve 820 is fully dilated (or expanded) under tension from the radial extension of the movable mechanical dilating structures 880.

Opposite movement of the handle of the mechanical sinus dilation device may then simultaneously retract the plurality of radially extending movable mechanical dilating structures 880 into a position substantially flush with the distal end of the mechanical sinus dilation device (see FIG. 7) and exert a positive pulling force via the one or more additional control structures 890 to pull back on the expandable dilation sleeve 820 as it compresses radially to substantially restore the mechanical sinus dilation device to its non-deployed, thinner configuration, substantially as shown in FIG. 7.

Those of skill in the art will recognize that the above discussion describes a single manner by which shrinkage/elongation of the expandable dilation sleeve 720,820 in an axial direction may be controlled. Alternatively, the expandable dilation sleeve 720,820 may be held in a non-deployed/non-dilated position by some manner of spring-like mechanism as the one or more additional control structures 790,890. Such a spring-like mechanism may be, for example, anchored more proximally (toward the handle) on an outer surface of an elongated thin body structure of the mechanical sinus dilation device (see FIG. 3), such that the expandable dilation sleeve 720,820 may be provided with a biasing force that would tend to facilitate retraction of the expandable dilation sleeve 720,820 in all conditions other than when being forced open, or radially expanded, by the radially extending movable mechanical dilating structures 780,880.

The exemplary embodiments of a mechanical sinus dilation device, as described above, substantially replace use of a conventional balloon/hydraulic mechanism for dilation, particularly of an inflamed, blocked and/or occluded eustachian tube, sinus passage, sinus cavity and/or sinus ostial opening. The disclosed embodiments describe a mechanical sinus dilation device that may instead use a positively-controlled mechanical dilation mechanism for expansion of a compatible and/or cooperating expandable dilation sleeve. An objective of the disclosed embodiments is to form a mechanical sinus dilation device with parts that can withstand exposure to extreme heat and pressure conditions, e.g., no plastic pieces, thereby rendering the mechanical sinus dilation device according to the disclosed embodiments autoclavable (sterilized by heat and pressure). Those of skill in the art recognize that any capacity by which to provide reusability in a sinus dilation device represents a distinct advantage in operation and in the marketplace. It should be recognized that the disclosed embodiments of the mechanical sinus dilation device may facilitate faster and easier use during surgery, and more positive control of the surgical procedure over a conventional balloon dilation device relying on inflation with water, and potentially requiring an additional person to accomplish the dilation procedure.

An advantage of the disclosed embodiments of the mechanical sinus dilation device resides in the distal dilation portion of the device being comprised of an external sleeve, which may be removable and replaceable, formed of a highly heat-resistant material or composition of materials including, but not limited to, a braided metal, Kevlar®, or similar material which would be strong, reusable, and which could be sterilized.

Another advantage of the disclosed embodiments of the mechanical sinus dilation device is represented by the operating mechanism in which the external sleeve may be positively activated and controlled with the deployment of radially extending rigid mechanical support members underneath the sleeve, in which radially extending components are raised from a substantially flush surface by movement of the surgeon's hand on the handle of the instrument.

In the described embodiments, activation of the radially extending rigid mechanical support members to the deployed position may occur through action of the surgeon's hand holding the instrument in a similar manner to other surgical instruments activated through manipulation of a standard two-ringed scissors-type handle. This particular description of a structure and operation of exemplary embodiments of the disclosed mechanical sinus dilation device is intended to be illustrative only and not limiting as to the many possible constructions and operations by which a distal end of such a mechanical sinus dilation device may be operated in the manner described.

In the described and depicted embodiments, again for illustration purposes only, the disclosed mechanical sinus dilation device may use an internal/central rod or wire within an outer body structure or sheath containing the radially extending rigid mechanical support members. This internal/central rod or wire may be connected to the radially extending rigid mechanical support members such that movement of the internal/central rod or wire may effectively deploy the radially extending rigid mechanical support members from the substantially flush position in the distal end of the device to radially expand the surrounding expandable dilation sleeve, as well as retract the radially extending rigid mechanical support members back to the flush position upon completion of the dilating procedure. The expandable dilation sleeve may substantially circumferentially surround the outer body structure or sheath containing the radially extending rigid mechanical support members. Deployment of the radially extending rigid mechanical support members may be effected in a manner whereby the expandable and circumferential expandable dilation sleeve is rigidly tented open by the radially extended rigid mechanical support members.

As described above, particularly with reference to FIGS. 7 and 8, embodiments of the disclosed mechanical sinus dilation device may include additional components for positively controlling rigid radial deployment/enlargement of the expandable dilation sleeve as well as potentially controlling shortening the expandable dilation sleeve during the dilation operation. In embodiments, the expandable dilation sleeve may be provided in a configuration such that adequate slack of the expandable dilation sleeve is available to facilitate the expandable dilation sleeve reaching full expansion under tension from the fully radially extended rigid mechanical support members. Structural components may be provided to actively control the shortening/elongation of the expandable dilation sleeve in a manner that activation of the handle of the device could simultaneously deploy both the radially extending rigid mechanical support members and shorten the expandable dilation, while opposite movement of the handle would retract the radially extending rigid mechanical support members and pull back on the expandable dilation sleeve to restore the distal portion of the mechanical sinus dilation device to its non-deployed, thinner configuration (see FIG. 7).

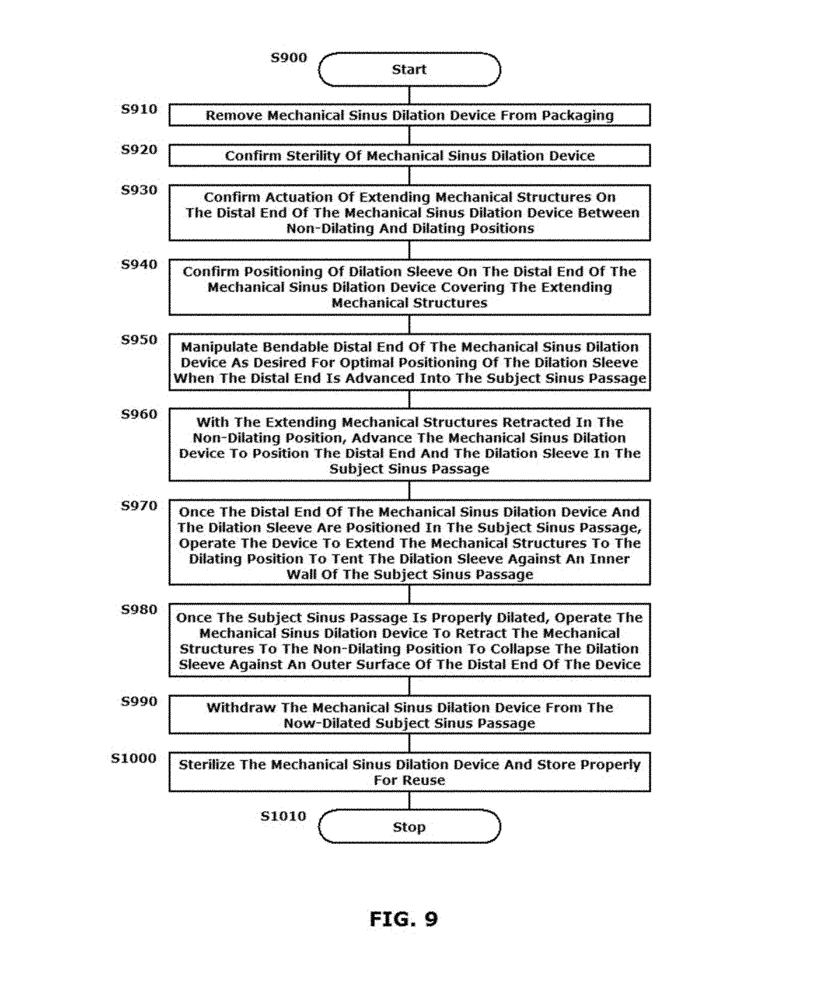

The disclosed embodiments may include a method for employing a reusable mechanical sinus dilation device in multiple sinus dilation procedures. FIG. 9 illustrates a flowchart of such an exemplary method. As shown in FIG. 9, operation of the method commences at Step S900 and proceeds to Step S910.

In Step S910, a mechanical sinus dilation device may be removed from its packaging. The packaging may be marked to advise of a sterilized condition of the mechanical sinus dilation device. Operation of the method proceeds to Step S920.

In Step S920, sterility of the mechanical sinus dilation device may be confirmed. Operation of the method proceeds to Step S930.

In Step S930, inspection may be undertaken to confirm actuation of extending mechanical structures on a distal end of the mechanical sinus dilation device between a first, flush or non-dilating position and a second, extended, or dilating position for the extending mechanical structures. Operation of the method proceeds to Step S940.

In Step S940, positioning of a dilation sleeve on the distal end of the mechanical sinus dilation device covering the extending mechanical structures may be confirmed. As detailed above, the dilation sleeve may be replaceable between operations. Additionally, the dilation sleeve may be substantially free-floating on the distal end of the mechanical sinus dilation device covering the extending mechanical structures, or may otherwise be fixedly attached to one or more additional mechanical structures, which may, for example, be employed to restrict, limit, or otherwise control an axial shrinking/elongation of the dilation sleeve in a dilation operation. Operation of the method proceeds to Step S950.

In Step S950, a bendable distal and of the mechanical sinus dilation device may be manipulated as desired for optimal positioning of the dilation sleeve when the distal end of the mechanical sinus dilation device is advanced into a patient's inflamed, blocked and/or occluded sinus passage, sinus cavity, and or sinus ostial opening (“the subject sinus passage”). Operation of the method proceeds to Step S960.

In Step S960, with the extending mechanical structures retracted in the non-dilating position, the mechanical sinus dilation device may be advanced to position the distal end, and the dilation sleeve, correctly in the subject sinus passage for effecting the dilation procedure. Operation of the method proceeds to Step S970.

In Step S970, once the distal end of the mechanical sinus dilation device, and the dilation sleeve, are confirmed to be positioned at an appropriate position in the subject sinus passage, an operating mechanism of the mechanical sinus dilation device may be operated, substantially in the manner described above, or in other like manner, to operate the mechanical sinus dilation device so as to extend the extending mechanical structures to the dilating position to tent the dilation sleeve against an inner wall of the subject sinus passage, thereby surgically dilating the subject sinus passage. Operation of the method proceeds to Step S980.

In Step S980, once the subject sinus passage is confirmed to have been properly dilated, the operating mechanism of the mechanical sinus dilation device may be operated to retract the extended mechanical structures to the non-dilating position (substantially flush with the outer surface of the distal end of the mechanical sinus dilation device) to collapse the dilation sleeve around the outer surface of the distal end of the mechanical sinus dilation device (see, e.g., FIG. 7). Operation of the method proceeds to Step S990.

In Step S990, the mechanical sinus dilation device may be withdrawn from the now-dilated subject sinus passage and the patient's nasal cavity. Operation of the method proceeds to Step S1000.

In Step S1000, the reusable mechanical sinus dilation device may be disassembled, as/if appropriate, cleaned and then sterilized and stored properly for reuse. Operation of the method proceeds to Step S1010, where operation of the method ceases.

The above-described exemplary systems, methods, apparatus, and/or devices reference certain conventional components, materials, and real-world use cases to provide a brief, general description of suitable operating and integration environments in which the subject matter of this disclosure may be implemented for familiarity and ease of understanding.

Those skilled in the medical field, and particularly in otolaryngology, will appreciate that other embodiments of the disclosed subject matter may be practiced in many disparate systems, procedures, techniques, processes and/or devices, including various structural components according to the embodiments described above to facilitate more efficient passage, cavity and/or opening dilation procedures.

The exemplary depicted sequence of method steps represents one example of a corresponding sequence of acts for implementing the functions described in the steps of the above-outlined exemplary method. The exemplary depicted steps may be executed in any reasonable order to carry into effect the objectives of the disclosed embodiments. No particular order to the disclosed steps of the method is necessarily implied by the depiction in FIG. 9, except where a particular method step is reasonably a necessary precondition to execution of any other method step.

Although the above description may contain specific details, they should not be construed as limiting the claims in any way. Other configurations of the described embodiments of the disclosed systems, methods, apparatus, devices, schemes and/or techniques are part of the scope of this disclosure.

It will be appreciated that various elements of the above-disclosed and other features and functions, or alternatives thereof, may be desirably combined into many other different systems or applications. Also, various alternatives, modifications, variations or improvements therein may be subsequently made by those skilled in the art which are also intended to be encompassed by the following claims.

We claim:

1. A dilation device, comprising:
a plurality of structural members disposed at a first axial end of a device, each of the plurality of structural members being operable between (1) a first position extending substantially axially along a structure of the device, and (2) a second position extending radially from the structure of the device;
a moving member extending axially along the structure of the device and being mechanically connected at a first axial end to at least some of the plurality of structural members, the moving member being axially displaceable with respect to the structure of the device to move the plurality of structural members between the first position and the second position; and
an expandable sleeve overlying the plurality of structural members at the first axial end of the structure of the device,
the expandable sleeve being radially expanded by the plurality of structural members in the second position.

2. The dilation device of claim 1, further comprising a plurality of actuating extensions,
at least a first of the plurality of actuating extensions being mechanically connected to the moving member and at least a second of the actuating extensions being mechanically connected to the structure of the device, and
axial displacement of the moving member with respect to the structure of the device being controlled by movement of the at least the first of the plurality of actuating extensions with respect to the at least the second of the plurality of the actuating extensions.

3. The dilation device of claim 2, the at least the first of the plurality of actuating extensions being a loop at a second axial end of the moving member, and
the axial displacement of the moving member with respect to the structure of the device being controlled by a plunger movement of the loop with respect to the at least the second of the plurality of the actuating extensions.

4. The dilation device of claim 2, further comprising:
a hollow body structure extending axially around at least a portion of the moving member, the plurality of structural members being disposed at a first axial end of hollow body structure;
a main body structure disposed at a second axial end of the hollow body structure,
the at least a second of the actuating extensions being mechanically connected to the hollow body structure via mechanical connection to the main body structure,
the at least the first of the plurality of actuating extensions mechanically connected to the moving member and the at least the second of the actuating extensions controlling the axial displacement of the moving member with respect to the hollow body structure by operating the at least the first of the plurality of actuating extensions in a scissor motion with respect to the at least the second of the plurality of the actuating extensions.

5. The dilation device of claim 1, further comprising at least one additional body structure mechanically connected at a first axial end to the expandable sleeve,
the at least one additional body structure limiting axial displacement of the expandable sleeve during radial expansion of the expandable sleeve.

6. The dilation device of claim 5, a second axial end of the at least one additional body structure being fixedly attached to the structure of the device proximal to the expandable sleeve.

7. The dilation device of claim 6, the at least one additional body structure being a spring.

8. The dilation device of claim 5, further comprising a hollow body structure extending axially around at least a portion of the moving member, the plurality of structural members and the expandable sleeve being disposed at a first axial end of hollow body structure,
the at least one additional body structure being a circumferential sleeve fixedly attached to an outer surface of the hollow body structure proximal to the expandable sleeve in a direction away from the first axial end.

9. The dilation device of claim 5, the at least one additional body structure being mechanically connected to the moving member and movable with respect to the structure of the device.

10. The dilation device of claim 9, movement of the at least one additional body structure being controlled to cooperate with movement of the moving member to positively control axial shortening and lengthening of the expandable sleeve during radial expansion and retraction.

11. The dilation device of claim 1, the first axial end of the structure of the device and the first axial end of the moving member being bendable with respect to the axis of the hollow body structure.

12. The dilation device of claim 1, an entire structure of the device being formed of materials that are heat resistant at temperatures exceeding 220° F., and at pressures greater than 12 psi.

13. The dilation device of claim 1, the expandable sleeve being formed of an expandable metal mesh.

14. The dilation device of claim 1, the expandable sleeve being formed of a pliable Kevlar® material.

15. The dilation device of claim 1, the expandable sleeve being removable.

16. The dilation device of claim 1, the first position of the plurality of structural members being a position in which an outer radial profile of each of the plurality of structural members is substantially flush with an outer surface of the structure of the device.

17. A method for dilation of a cavity, comprising:
providing a dilation device having
a first body structure;
a plurality of structural members disposed at a first axial end of the first body structure, each of the plurality of structural members being operable between (1) a first position extending substantially axially along the first body structure, and (2) a second position extending radially from the first body structure;
a moving member extending axially with respect to the first body structure and being mechanically connected at a first axial end to at least some of the plurality of structural members, the moving member being axially displaceable with respect to the first body structure to move the plurality of structural members between the first position and the second position; and
an expandable sleeve overlying the plurality of structural members at the first axial end of the first body structure;
advancing the first axial end of the first body structure of the dilation device into a cavity;
operating the dilation device by axially displacing the moving member with respect to the first body structure to radially extend the plurality of structural members from the first position to the second position to radially expand the expandable sleeve against an inner wall of the cavity.

18. The method of claim 17, the dilation device further comprising a plurality of actuating extensions,
at least a first of the plurality of actuating extensions being mechanically connected to the moving member and at least a second of the actuating extensions being mechanically connected to the first body structure,
the operating the dilation device further comprising moving the at least the first of the plurality of actuating extensions with respect to the at least the second of the plurality of the actuating extensions.

19. The method of claim 18, the at least the first of the plurality of actuating extensions being a loop at a second axial end of the moving member,
the moving the at least the first of the plurality of actuating extensions with respect to the at least the second of the plurality of the actuating extensions comprising a plunger movement of the loop with respect to the at least the second of the plurality of the actuating extensions.

20. The method of claim 18, the dilation device further comprising a second body structure disposed at a second axial end of the first body structure,
the at least a second of the actuating extensions being mechanically connected to the first body structure via mechanical connection to the second body structure,
the moving the at least the first of the plurality of actuating extensions with respect to the at least the second of the plurality of the actuating extensions comprising operating the at least the first of the plurality of actuating extensions in a scissor motion with respect to the at least the second of the plurality of the actuating extensions.
